(19) United States
(12) Patent Application Publication
(10) Pub. No.: US 2011/0306576 A1
(43) Pub. Date: Dec. 15, 2011

(54) COMPOUNDS AND METHODS RELATING THERETO

(75) Inventor: Mark Wainwright, Lancashire (GB)

(73) Assignee: PHARMALUCIA LIMITED, Merseyside (GB)

(21) Appl. No.: 13/203,512

(22) PCT Filed: Feb. 25, 2010
(86) PCT No.: PCT/GB10/50325

§ 371 Date: Aug. 26, 2011

(30) Foreign Application Priority Data
Feb. 27, 2009 (GB) 0903348.1

(51) Int. Cl. A61K 31/5415; C12Q 1/02; C07D 279/20; C07F 7/18; C07D 417/12; A61L 2/00; A61K 31/695; A61P 31/12; A61P 31/04; A61P 31/10; A61P 33/02; C12Q 1/70; C07D 417/14

(52) U.S. Cl. 514/63; 435/5; 435/29; 544/37; 514/224.8; 514/225.2; 514/225.8; 422/22

(57) ABSTRACT

A compound of formula (II) or a pharmaceutically acceptable derivative thereof for use in a method of combating and/or detecting a pathogen; wherein X is selected from O, S and Se; each of R¹, R², R⁴, R⁶, R⁸, R⁹, R¹¹, R¹², R¹³ and R¹⁴ is independently selected from hydrogen, halogen, sulfo, sulfoxy, mercapto, acyl, nitro, amino or an optionally substituted alkyl, alkenyl, alkynyl, aryl, amine or alkoxy group; provided that at least one of R¹¹, R¹², R¹³ and R¹⁴ is an optionally substituted amine group.

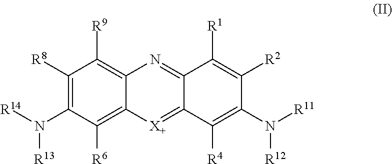

The present invention relates to novel compounds, compositions comprising these and other compounds, and method and uses relating thereto. In particular the present invention relates to photosensitising compounds, especially those which induce the formation of reactive oxygen species on exposure to light of a particular wavelength. The compounds of the present invention have been found to be useful in methods for combating pathogens, especially viruses, and in methods for detecting pathogens.

Methylene blue has the structure shown in formula (I) and has been known as a photosensitising antiviral agent since the 1930s.

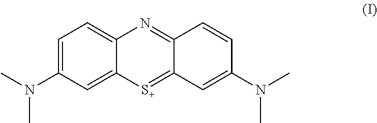

However the use of methylene blue as a virucidal agent has some disadvantages, for example in terms of collateral effects to host cells and thus there is a continuing need to provide improved antiviral agents.

The present invention seeks to provide compounds and compositions having improved properties.

According to a first aspect of the present invention there is provided a compound of formula (II):

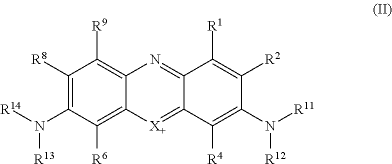

or a pharmaceutically acceptable derivative thereof for use in a method of combating and/or detecting a pathogen;
wherein X is selected from O, S and Se; each of R¹, R², R⁴, R⁶, R⁸, R⁹, R¹¹, R¹², R¹³ and R¹⁴ is independently selected from hydrogen, halogen, sulfo, sulfoxy, mercapto, acyl, nitro, amino or an optionally substituted alkyl, alkenyl, alkynyl, aryl, amine or alkoxy group; provided that at least one of R¹¹, R¹², R¹³ and R¹⁴ is an optionally substituted amine group.

Pharmaceutically acceptable derivatives include salts and solvates, such as acid addition salts. Thus the compounds of the invention may include an anion, monovalent or polyvalent, sufficient to balance the charge on the compound of formula (II). Examples of suitable counter ions include both organic and inorganic moieties. Suitable organic moieties include acetate, citrate and tartrate. Suitable inorganic moieties include halide, carboxylate, sulphate and phosphate counterions. Especially preferred counterions are sulphate and chloride.

In some preferred embodiments X is sulphur or selenium, most preferably sulphur.

In some preferred embodiments, X is oxygen.

When any of R¹, R², R⁴, R⁶, R⁸, R⁹, R¹¹, R¹², R¹³ or R¹⁴ is an alkyl group, it is preferably an alkyl group having from 1 to 30 carbon atoms, preferably 1 to 20 carbon atoms, more preferably 1 to 12, for example 1 to 8 carbon atoms, preferably 1 to 6 carbon atoms and most preferably from 1 to 4 carbon atoms.

The or each such alkyl group may be straight chained or branched and may be optionally substituted with one or more substituents selected from halogen, nitro, sulfoxy, sulfo, amino, acyl, hydroxy, alkoxy (especially C1 to C4 alkoxy), mercapto or a silicon containing group.

When any of R¹, R², R⁴, R⁶, R⁸, R⁹, R¹¹, R¹², R¹³ or R¹⁴ is an alkenyl group, it is preferably an alkenyl group having from 2 to 30 carbon atoms, preferably 2 to 20 carbon atoms, more preferably 2 to 12, for example 2 to 8, preferably 2 to 6 carbon atoms and most preferably from 2 to 4 carbon atoms.

The or each such alkenyl group may be straight chained or branched and may have an E or a Z configuration. Each such alkenyl group may be optionally substituted with one more substituents selected from halogen, nitro, sulfo, sulfoxy, amino, acyl, hydroxy, alkoxy (especially C1 to C4 alkoxy), mercapto or a silicon containing group.

When any of R¹, R², R⁴, R⁶, R⁸, R⁹, R¹¹, R¹², R¹³ or R¹⁴ is an alkynyl group, it is preferably an alkynyl group having from 2 to 30 carbon atoms, preferably 2 to 20 carbon atoms, more preferably 2 to 12, for example 2 to 8 carbon atoms, preferably 2 to 6 carbon atoms and most preferably 2 to 4 carbon atoms.

The or each such alkynyl group may be straight chained or branched and may be optionally substituted with one or more substituents selected from halogen, nitro, sulfo, sulfoxy, amino, acyl, hydroxy, alkoxy (especially C1 to C4 alkoxy), mercapto or a silicon containing group.

When any of R¹, R², R⁴, R⁶, R⁸, R⁹, R¹¹, R¹², R¹³ or R¹⁴ is an aryl group, it may be an aryl group comprising only carbon atoms within the aromatic ring or it may be a heteroaromatic moiety including one or more heteroatoms selected from nitrogen, sulphur and oxygen. Preferably the or each aryl group has between 3 and 15 atoms in the aromatic ring, preferably between 5 and 10 atoms. The or each such aryl group may be optionally substituted with one or more substituents selected from halogen, nitro, sulfo, sulfoxy, amino, hydroxyl, acyl, alkoxy (especially C1 to C4 alkoxy), alkyl (especially C1 to C5 alkyl), mercapto or a silicon containing group.

Preferably when any of R¹, R², R⁴, R⁶, R⁸, R⁹, R¹¹, R¹², R¹³ or R¹⁴ is an aryl group it is an aryl group comprising only carbon atoms in the aromatic ring. Preferably it comprises from 6 to 10 carbon atoms.

When any of R¹, R², R⁴, R⁶, R⁸, R⁹, R¹¹, R¹², R¹³ or R¹⁴ is an alkoxy group, it is preferably an alkoxy group having from 1 to 30 carbon atoms, preferably 1 to 20 carbon atoms, more preferably 1 to 12, for example 1 to 8 carbon atoms, preferably 1 to 6 carbon atoms and most preferably 1 to 4 carbon atoms. Each alkyl group of an alkoxy substituent may be straight chained or branched and may be substituted with one or more substituents selected from halogen, nitro, amino, acyl, hydroxy, alkoxy (especially C1 to C4 alkoxy), mercapto or a silicon containing group.

Suitable silicon containing groups are those of formula SiA₃ in which each A is independently selected from an optionally substituted alkyl, alkenyl, alkynyl or alkoxy group. Any such optionally substituted alkyl, alkenyl, alkynyl or alkoxy group may be as defined in relation to R¹, R², R⁴, R⁶, R⁸, R⁹, R¹¹, R¹², R¹³ or R¹⁴ above. Preferably each A is independently selected from optionally substituted alkyl or alkoxy groups. More preferably each is independently selected from unsubstituted alkyl or alkoxy groups, preferably those having 1 to 10 carbon atoms, more preferably 1 to 6 carbon atoms, for examples 1 to 4 carbon atoms.

Optionally substituted alkyl, alkenyl, alkynyl or alkoxy groups may be substituted along the length of the chain and/or at the end of the chain to include a terminal substituent. Alternatively and/or additionally each or any of these groups may be substituted within the chain with one or more hetero atoms, for example oxygen, nitrogen or sulphur to form an ether, thioether or amino linkage, or silicon. In embodiments in which the alkyl, alkenyl, alkynyl or alkoxy group includes a silicon atom in the chain, this may be directed bonded to two carbon atoms of the chain or it may be bonded via an oxygen linkage to form a silyl ether within the chain. In addition to the groups forming part of the alkyl, alkenyl, alkynyl or alkoxy chain, the silicon atom will be bonded to two further groups, or three further groups in the case of a terminal silicon substituent. These further groups may comprise any suitable group as would be known to the person skilled in the art. However in preferred embodiments the further groups are independently selected from optionally substituted alkyl or alkoxy groups. More preferably they are independently selected from unsubstituted alkyl or alkoxy groups, preferably those having 1 to 10 carbon atoms, more preferably 1 to 6 carbon atoms, for example 1 to 4 carbon atoms.

When any of R¹, R², R⁴, R⁶, R⁸, R⁹, R¹¹, R¹², R¹³ or R¹⁴ is an optionally substituted amine group, it may be an alkyl amine, alkenyl amine, alkynyl amine, aryl amine or alkoxy amine moiety. Any of R¹, R², R⁴, R⁶, R⁸, R⁹, R¹¹, R¹², R¹³ or R¹⁴ may by a group of formula R¹⁵NR¹⁶R¹⁷ wherein R¹⁵ is selected from a bond, or an optionally substituted alkylene, alkenylene, alkynylene or arylene moiety and each of R¹⁶ and R¹⁷ is independently selected from hydrogen or an optionally substituted alkyl, alkenyl, alkynyl, aryl or alkoxy residue. Preferably R¹⁵ is selected from an optionally substituted alkylene or alkenylene group, most preferably an optionally substituted alkylene group, especially an alkylene group having 1 to 12 carbon atoms, preferably 1 to 10, for example 1 to 8, more preferably 1 to 6 and most preferably 1 to 4 carbon atoms, for example 2 or 3 carbon atoms. Such an alkylene group be straight chained or branched. It may be substituted with one or more substituents selected from halogen, nitro, amino, acyl, hydroxy, alkoxy (especially C1 to C4 alkoxy), mercapto or a silicon containing group (as previously defined herein). Preferably it is unsubstituted. Preferably it is not branched.

Each of R¹⁶ and R¹⁷ may be independently selected from hydrogen, and an optionally substituted alkyl, alkenyl, alkynyl, aryl or alkoxy group. Preferably each is independently selected from an optionally substituted alkyl or alkenyl group, preferably having up to 30 carbon atoms, preferably up to 24 carbon atoms, for example 18 carbon atoms, more preferably up to 12 carbon atoms, preferably up to 8 carbon atoms, more preferably up to 6 carbon atoms. Such optionally substituted alkyl or alkenyl groups may be straight chained or branched. Preferably they are straight chained. When either or each of R¹⁶ or R¹⁷ is an alkyl group it is preferably an alkyl group having 1 to 4 carbon atoms, for example methyl, ethyl, propyl (especially isopropyl) or butyl (especially tertiary butyl). In embodiments in which R¹⁶ and/or R¹⁷ is alkenyl, each is preferably an alkenyl group having 2 to 4 carbon atoms. In embodiments in which R¹⁶ and/or R¹⁷ is an alkenyl group, each double bond may have an E or a Z configuration.

In some embodiments R⁹ and R⁹ may be linked to form a ring. They may form an aromatic ring or an aliphatic ring. Suitably in such embodiments R⁸ and R⁹ are linked to form a ring including from 5 to 7 atoms (the total number of atoms in the ring). The ring may include one or more hetero atoms within the ring for example O, S, N or Si. The ring may be optionally substituted for example with one or more substituents selected from halogen, nitro, sulfo, sulfoxy, amino, acyl, hydroxy, mercapto, alkoxy (especially C1 to C4 alkoxy) mercapto or a silicon containing group (as previously defined herein).

In some preferred embodiments R⁸ and R⁹ are joined to form a 6-membered ring. Especially preferred is an aromatic 6-membered ring, for example a benzene ring.

In some embodiments R¹ and R² may be linked to form a ring. They may form an aromatic ring or an aliphatic ring. Suitably in such embodiments R¹ and R² are linked to form a ring including from 5 to 7 atoms (the total number of atoms in the ring). The ring may include one or more hetero atoms within the ring for example O, S, N or Si. The ring may be optionally substituted for example with one or more substituents selected from halogen, nitro, sulfo, sulfoxy, amino, acyl, hydroxy, mercapto, alkoxy (especially C1 to C4 alkoxy) mercapto or a silicon containing group (as previously defined herein).

In some preferred embodiments R¹ and R² are joined to form a 6-membered ring. Especially preferred is an aromatic 6-membered ring, for example a benzene ring.

The present invention may include compounds of formula (II) in which R¹¹ or R¹² together with R² form a ring and/or compounds in which R¹³ or R¹⁴ together with R⁹ form a ring. Preferably however the present invention does not include compounds in which R¹¹ or R¹² is joined with R² to form a ring or R¹³ or R¹⁴ is joined with R⁹ to form a ring.

Preferably each of R¹, R², R⁸ and R⁹ is independently selected from hydrogen, an optionally substituted alkyl group or an optionally substituted alkoxy group.

Preferably when any of R¹, R², R⁸ or R⁹ is an alkyl group it is an alkyl group having from 1 to 10, preferably 1 to 6, most preferably 1 to 4 carbon atoms. Thus each of R¹, R², R⁸ and R⁹ may be independently selected from methyl, ethyl, propyl (especially isopropyl), and butyl (especially tertiary butyl).

Preferably when any of R¹, R², R⁸ and R⁹ is an alkoxy group, it is an alkoxy group having from 1 to 10, preferably 1 to 6, more preferably 1 to 4 carbon atoms. Suitably each of R¹, R², R⁸ and R⁹ may be independently selected from methoxy, ethoxy, propoxy (especially isopropoxy) or butoxy (especially tertiary-butoxy).

When any of R¹, R², R⁸ or R⁹ is alkyl or alkoxy, the alkyl group may be straight-chained or branched. Any such group may be optionally substituted with one or more groups selected from halogen, amino, hydroxyl, acyl, sulfo, sulfoxy, mercapto, alkoxy (especially C1 to C4 alkoxy) or a silicon containing group (as previously defined herein). A substituent may be along the alkyl chain of the alkyl or alkoxy group or at the end of the chain. In some preferred embodiments, a terminal hydroxy or amino group is present. Thus each of R¹, R², R⁸ or R⁹ may be independently selected from a group of formula —O(CH₂)ₙOH or —(CH₂)ₙOH where n is from 1 to 10, preferably 1 to 6, for example 2 to 4.

In preferred embodiments each of R⁴ and R⁶ is independently selected from hydrogen and halogen. When either or each of R⁴ and R⁶ is halogen, each may independently be fluorine, chlorine, bromine or iodine. When X is S or Se and R⁴ and/or R⁶ is halogen, bromide or chlorine is preferred. When X is O, iodine is preferred.

The present invention relates to compounds of formula (II) in which at least one of R¹¹, R¹², R¹³ and R¹⁴ is an optionally substituted amine group.

In this specification the term “optionally substituted amine group” is used to refer to a group of formula R₃N in which at least one R group is not hydrogen. It also includes quaternary ammonium salts of formula R₄N⁺ in which at least one R is not hydrogen.

In some embodiments each of R¹¹, R¹², R¹³ and R¹⁴ is an optionally substituted amine group.

In some embodiments at least one of R¹¹ and R¹² is an optionally substituted amine group and at least one of R¹³ and R¹⁴ is an optionally substituted amine group.

In some embodiments each of R¹¹ and R¹² is an optionally substituted amine group.

In some embodiments each of R¹³and R¹⁴ is an optionally substituted amine group.

In some embodiments one of R¹¹ and R¹² is an optionally substituted amine group and one of R¹³ and R¹⁴ is an optionally substituted amine group.

In some especially preferred embodiments, any or each of R¹¹, R¹², R¹³ and R¹⁴ which is an optionally substituted amine group may be a quaternary amine salt. Some compounds of formula (II) may be protonated at physiological pH and will thus exist as the quaternary ammonium salt but under basic conditions will reform the free amine. However compounds including a permanent cation are also within the scope of the present invention.

Thus each of R¹¹, R¹², R¹³, and R¹⁴, may be independently selected from a group of formula R¹⁵NR¹⁶R¹⁷ or R¹⁵N⁺R¹⁶R¹⁷R¹⁸ wherein each of R¹⁵, R¹⁶ and R¹⁷ is suitably as defined above, and R¹⁸ is hydrogen or an optionally substituted alkyl, alkenyl, alkynyl, aryl or alkoxy group.

In especially preferred embodiments R¹⁵ is an alkylene group having 1 to 8, especially 1 to 6 carbon atoms, more preferably 1 to 4 carbon atoms, for example 2 or 3 carbon atoms, for example 2 or 3 carbon atoms.

In especially preferred embodiments, each of R¹⁶ and R¹⁷ is independently selected from hydrogen and an alkyl group having 1 to 10, preferably 1 to 8, suitably 1 to 6, for example 1 to 4 carbon atoms, for example 2 or 3 carbon atoms.

Preferably R¹⁸ when present is selected from hydrogen or an optionally substituted alkyl group. Such an alkyl group may be straight-chained or branched and may be substituted with one or more substituents selected from halogen, nitro, sulfo, sulfoxy, hydroxyl, acyl, alkoxy (especially C1 to C4 alkoxy), amino, mercapto mercapto or a silicon containing group (as previously defined herein).

Preferably R¹⁸ is an unsubstituted group. In especially preferred embodiments R¹⁸ is an alkyl group having 1 to 10 carbons, preferably 1 to 6 carbon atoms, more preferably 1 to 4 carbons. Most preferably R¹⁸ selected from methyl or ethyl.

Suitably one or more of R¹¹, R¹², R¹³ and R¹⁴ may include a quaternary ammonium group. Thus the compound of formula (II) may include none, one, two, three or four quaternary ammonium groups. The resultant “molecule” may then respectively have an overall charge of +1, +2, +3, +4 or +5. The skilled person will appreciate that by the term “molecule” we mean to refer to the structure shown in figure (II) and there will of course be present a suitable number of anionic counterions.

Each of R¹¹, R¹², R¹³ and R¹⁴ which is not an optionally substituted amine group is preferably independently selected from hydrogen and an optionally substituted alkyl, alkenyl, alkynyl, alkoxy or aryl group. Most preferably each of R¹¹, R¹², R¹³ and R¹⁴ which is not an optionally substituted amine group is independently selected from hydrogen and an optionally substituted alkyl group.

Any such alkyl group preferably has from 1 to 12, preferably 1 to 8, more preferably 1 to 6, especially 1 to 4 carbon atoms. Suitably any such alkyl group may be selected from methyl, ethyl, propyl (especially isopropyl) and butyl (especially tert-butyl).

The or each such alkyl group may be optionally substituted with one or more groups selected from halogen, amino, hydroxy, sulfo, sulfoxy, mercapto, acyl, alkoxy (especially C1 to C4 alkoxy), mercapto or a silicon containing group (as previously defined herein).

In some embodiments R¹⁶ and R¹⁷ may together form a ring, preferably an aliphatic ring, suitably a ring having from 3 to 8 atoms, preferably 5 to 7 atoms, including the nitrogen atom, and optionally further heteroatoms within the ring. For example R¹⁶ and R¹⁷ may together form aliphatic ring selected from pyrrolidine, pyrroline, imidazoline, oxazolidine, thiazolidine, piperidine, azepine, oxazine morpholine, thiomorpholine and piperazine. When the heterocyclic ring includes a further nitrogen atom, this may have a further alkyl group bonded thereto. This alkyl group and/or atoms within the ring may be optionally substituted with one or more substituents selected from halogen, amino, nitro, sulfo, sulfoxy, acyl, alkoxy (especially C1 to C4 alkoxy), hydroxy, mercapto mercapto or a silicon containing group (as previously defined herein).

In some embodiments the present invention relates to the use of compounds of formula (II) in which one of R¹¹, R¹², R¹³ or R¹⁴ is a group of formula (III)

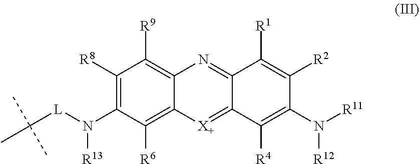

wherein L is a linking group and R¹, R², R⁴, R⁶, R⁸, R⁹, R¹¹, R¹² and R¹³ are as previously defined herein.

Thus the compounds of the present invention may be provided as dimer.

Preferably L is an optionally substituted alkylene, alkenylene, alkynylene or arylene moiety and may for example include a carbonyl group such that an amide linkage is formed, or an additional amine functionality. Preferably L is an alkylene group, preferably an alkylene group having 1 to 10, preferably 1 to 6, suitably 1 to 4 for example 2 or 3 carbon atoms. In some preferred embodiments it is an unsubstituted alkylene group.

Thus in some preferred embodiments, the present invention relates to compounds of formula (IV):

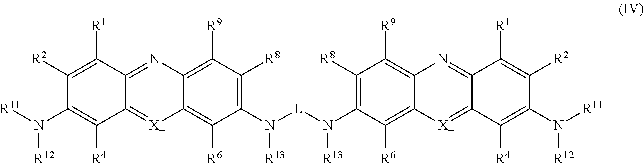

wherein L is a suitable linking group and each of X, R¹, R², R⁴, R⁶, R⁸, R⁹, R¹¹, R¹² and R¹³ is independently selected from those previously defined herein.

The first aspect of the present invention provides compounds for use in a method of combating and/or detecting a pathogen.

The pathogen may be selected from viruses, bacteria, fungi and protozoal infections.

In some preferred embodiments the first aspect of the present invention provides compounds of formula (II) for use in a method of combating and/or detecting bacteria.

Suitably the present invention provides compounds for use in a method of combating and/or detecting a bacteria selected from Gram positive bacteria, Gram negative bacteria and mycobacteria.

By a method of combating bacteria we mean to include methods which kill bacteria and/or methods which inhibit the growth of bacteria and/or methods which prevent the growth of bacteria.

The compounds of formula (II) may be regarded as antibacterial agents. Thus the present invention further provides compounds of formula (II) for use as an antibacterial agent.

In some preferred embodiments the first aspect of the present invention provides compounds of formula (II) for use in a method of combating and/or detecting fungi.

Suitably the present invention provides compounds for use in a method of combating and/or detecting a fungus selected from, but not limited to candida, aspergillus, cryptococcus, histoplasma, pneumocystis and stachybotrys.

By a method of combating fungi bacteria we mean to include methods which kill fungi and/or methods which inhibit the growth of fungi and/or methods which prevent the growth of fungi.

The compounds of formula (II) may be regarded as antifungal agents. Thus the present invention further provides compounds of formula (II) for use as an antifungal agent.

The present invention may provide a method of combating and/or detecting protazoal species which can cause infection.

By a method of combating protazoal species we mean to include methods which kill protazoal species and/or methods which inhibit the growth or protazoal species and/or methods which prevent the growth of protazoal species.

Suitably the present invention provides compounds for use in combating and/or detecting a protazoal species selected from plasmodia, leishmania and trypanosoma.

In an especially preferred embodiment, the first aspect of the present invention provides compounds of formula (II) for use in a method of combating and/or detecting a virus.

Suitably the present invention provides compounds for use in a method of combating and/or detecting a virus selected human immunodeficiency viruses, common cold viruses, influenza, herpes, hepatitis and West Nile viruses.

By a method of combating a virus we mean to include methods which kill a virus and/or methods which inhibit the growth of a virus and/or methods which prevent the growth of a virus.

The compounds of formula (II) may be regarded as antiviral agents. Thus the present invention further provides compounds of formula (II) for use as an antiviral agent.

Compounds effective for combating a virus include those compounds of formula (II) in which X is S or Se.

The compounds used in the present invention are photosensitising and it is believed that the anti-pathogenic activity is achieved upon exposure to light. Exposing the compounds to light of a specific wavelength suitably causes the formation of reactive oxygen species, for example singlet oxygen, hydroxyl radicals or superoxide.

By photosensitising we mean to refer to compounds which are reactive to light, especially light of a particular wavelength, but do not necessarily decompose upon exposure to light.

Thus the present invention provides compounds of formula (II) for use in a method of combating and/or detecting a pathogen wherein the method comprises exposing the compounds to light of an appropriate wavelength in the presence of oxygen.

It is believed that the singlet oxygen produced upon irradiation with light is involved in combating a pathogen.

In some embodiments irradiation with light does not lead to the formation of singlet oxygen but does cause the compound to fluoresce. In such embodiments the compounds may be used in a method of detecting a pathogen. Preferred compounds for use in detecting a pathogen are those of formula (II) in which X is O. Especially useful in detection methods are compounds in which X is O.

Suitably the compounds of formula (II) are activated upon exposure to light having a wavelength of from 500 to 900 nm, preferably 550 to 850 nm, for example 600 to 750 nm, more preferably 620 to 700 nm. Light of the desired wavelength may suitably be provided by an LED or a laser.

Without wishing to be bound by theory it is believed that the phenothiazinium moiety of the compounds of formula (II) is able to intercalate between the DNA of viruses. Irradiation with light produces singlet oxygen which then damages the DNA. It is believed that the inclusion of basic side chains helps anchor the compounds of formula (II) to the nucleic acid backbone in viruses. The killing process in other microbial types may include action against DNA, cell wall or other organelles.

In some embodiments the compounds of the present invention may be used in a method of combating and/or detecting a pathogen, especially a virus, in a blood product. Suitably the blood product is contacted with the compound of formula (II) and then irradiated with light of an appropriate wavelength, for example 600 to 800 nm. Thus the present invention suitably provides a method of reducing the level of a pathogenic contaminant in a blood product.

By a blood product we mean to refer to whole blood or a component of whole blood, for example cellular blood components (including red blood cells and platelets), blood proteins (for example blood clotting factors, enzymes, albumin, plasminogen, and immunoglobins) and liquid blood components (for example plasma and plasma-containing compositions).

The present invention may provide a method of combating pathogens in a blood product. It may also provide a method of detecting pathogenic species, especially a virus in a blood product. In such a detection method irradiation with light of a particular wavelength enables the compound of formula (II) to be seen but it does not produce singlet oxygen.

In some embodiments the compounds of formula (II) are included in a composition suitable for administration to a patient infected with a virus.

According to a second aspect of the present invention there is provided a composition comprising a compound of formula (II) as defined in relation to the first aspect and a pharmaceutically acceptable carrier.

The composition of the second aspect may be provided in any suitable form depending upon the desired application. Preferably the composition is provided in a form suitable for topical application. For example it may be provided in the form of a gel, paste, lotion, powder, solution, cream or the like.

The composition of the present invention may comprise any suitable pharmaceutically acceptable carrier.

The pharmaceutically acceptable carrier may be aqueous based or it may be based on an organic solvent. It may comprise water, an organic solvent or a mixture thereof. In preferred embodiments the compound of formula (II) is completely dissolved in the carrier.

Preferred organic solvents for inclusion in the carrier are water miscible solvents, for example an alcohol such as a C1 to 4 alcohol (methanol, ethanol, n-proponol, isoproponol, n-butanol and sec-butanol); amides; ketones (for example acetone and methyl and ether ketone); water miscible ethers; and diols (especially diols having 1 to 12 carbon atoms, for example ethylene glycol).

Suitably the composition comprises from 0.0001 to 50 wt % of one or more compounds of formula (II), preferably from 0.005 to 20 wt %, preferably from 0.01 to 15 wt %, more preferably from 0.005 to 10 wt %, for example from 0.1 to 5 wt %.

When a composition of the present invention is used topically to treat a viral infection, the composition is suitably applied to the infected area and then light of the appropriate wavelength is applied. This will typically be carried out by a skilled healthcare professional who is appropriately trained. The composition may be used in a similar manner to topically treat a bacterial or fungal infection or an infection caused by a protazoal species.

Although the compounds of the present invention are preferably provided in a composition suitable for external topical use, alternative methods in which the compounds can be delivered to the targeted sites and irradiated are also within the scope of the invention.

According to a third aspect of the present invention, there is provided compounds of formula (II), other than compounds having the formula Va to Ve:

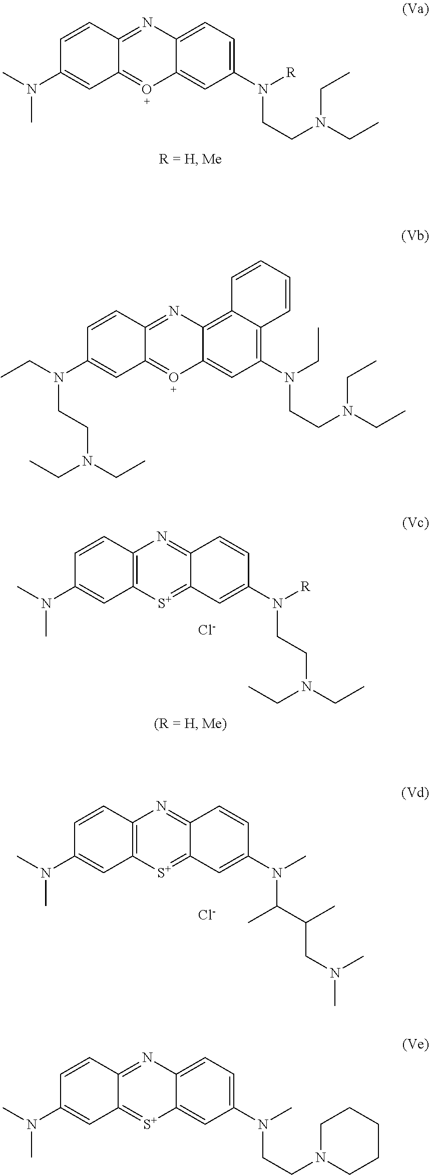

Preferred features of the second and third aspects of the present invention are as defined in relation to the first aspect.

Especially preferred compounds of the present invention are shown in figure (VI):

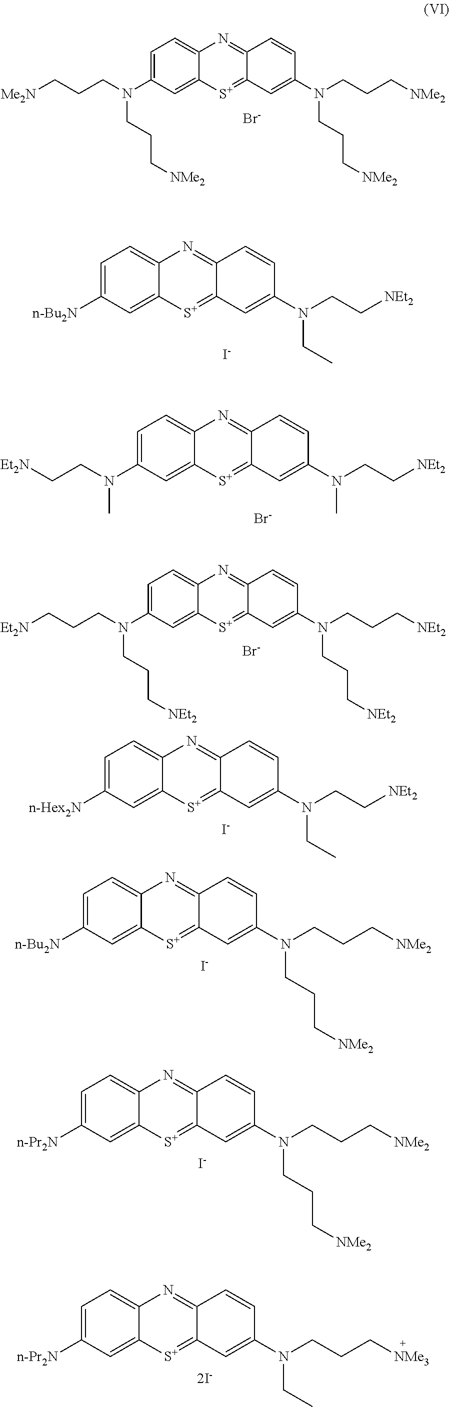

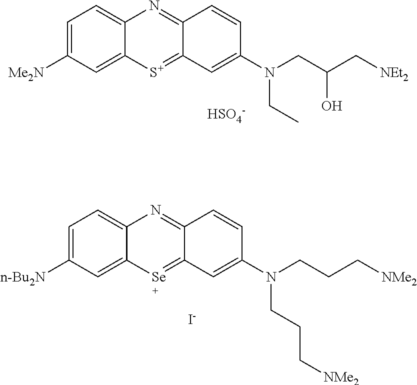

According to a fourth aspect of the present invention there is provided a method of preparing a compound of formula (II), the method comprising reacting a phenothiazinium compound of formula (VII):

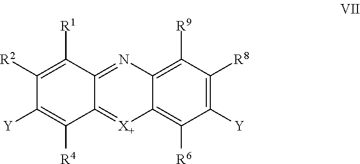

with an amine of formula R¹³R¹⁴NH and an amine of formula R¹¹R¹²NH;
wherein R¹, R², R⁴, R⁶, R⁸, R⁹, R¹¹, R¹², R¹³ and R¹⁴ are as defined in relation to the first aspect and each Y is independently a halogen or hydrogen.

Preferably each Y is independently hydrogen or bromine. Preferably each Y is the same.

In embodiments in which each Y is bromine, R¹¹R¹²NH is preferably the same as R¹³R¹⁴NH.

In embodiments in which each Y is hydrogen, R¹¹R¹²NH may be the same as R¹³R¹⁴NH or it may be different. In embodiments in which R¹¹R¹²NH and R¹³R¹⁴NH are different they are suitably added sequentially. Otherwise they may be added sequentially or simultaneously.

The compound of formula (VII) by be prepared by a known method from 10H-pheonthiazine, which is a commercially available compound.

For example, the compounds of formula (VII) in which Y is hydrogen may be prepared from 10H-phenothiazine by reaction with iodine. Reaction may suitably be carried out in a chlorinated solvent (for example chloroform) at room temperature to provide the compound of formula (VII) as the tetraiodine salt. Compounds of formula (VII) in which Y is Br may be prepared by treating 10H-phenothiazine with bromine. The reaction may sutably be carried out in a protic solvent (for example acetic acid) at room temperature to provide the compound of formula (VII) as the bromide salt.

In the method of the fourth aspect of the present invention, the compounds of formula (VII) are reacted with one or more amines. At least 2 molar equivalents of amine to the compound of formula (VII) is needed. Preferably a molar excess of amine is used.

Where R¹¹R¹²NH and R¹³R¹⁴NH are the same a large excess of amine may be used, for example 5 to 20 molar equivalents, preferably about 10 equivalents.

Where R¹¹R¹²NH and R¹³R¹⁴NH are different, preferably the compound of formula (VII) is first reacted with from 1.5 to 5, for example approximately 2.5 equivalents of the amine of formula R¹¹R¹²NH and the product is subsequently reacted with 2 to 10, for example approximately 5 equivalents of the amine of formula R¹³R¹⁴NH.

The reactions of the compounds of formula (VII) with an amine are suitably carried out at room temperature. Suitable solvents include alcohols, for example methanol, and halogenated solvents, for example dichloromethane. Typical reaction times are from 0.5 to 12 hours, suitably 1 to 6 hours, for example 2 to 3 hours.

The above reaction conditions are merely illustrative and the person skilled in the art would be able to vary these as appropriate.

According to a fifth aspect of the present invention there is provided a method of treating an animal having a disease caused by a pathogen, the method comprising administering to the animal a compound of formula (II). Preferably the method of the fifth aspect comprises administering to the animal a composition of the second aspect.

Preferably the animal is a mammal. More preferably the animal is a human.

Preferred features of the fifth aspect are as defined in relation to the first, second, third and fourth aspects.

According to a sixth aspect of the present invention there is provided a method of combating and/or detecting a pathogen at a locus, the method comprising applying to the locus a composition comprising a compound of formula (II), and exposing the locus to light having a wavelength of from 500 to 900 nm.

Preferred features of the sixth aspect are as defined in relation to the first, second, third, fourth and fifth aspects.

The invention will now be further described with reference to the following non-limiting examples.

### EXAMPLE 1

### Preparation of Arylthiosulphonic Acids: 2-Amino-5-dialkylaminophenylthiosulphonic acid

N,N-Dialkyl-p-phenylenediamine sulphate (130 mmol) was added to a mechanically stirred solution of aluminium sulfate octadecahydrate/water (43.6 g, 65 mmol/100 ml). To this was added sodium thiosulfate/water (22.0 g, 139 mmol/80 ml) followed by zinc chloride/water (8.8 g, 63 mmol/12 ml). The solution was cooled to 0° C. and potassium dichromate/water (5.0 g, 17 mmol/20 ml) was added dropwise over a 30 minute period. Following this addition, the mixture was allowed to stir for 2 hours. During the last 30 minutes the temperature was allowed to rise to 10° C. causing the formation of a viscous precipitate. This was isolated by filtration and washed with water followed by acetone.

### EXAMPLE 1a

2-Amino-5-dimethylaminophenylthiosulphonic acid was prepared as above from N,N-dimethyl-p-phenylenediamine sulphate, yield=15.87 g (49%), m.p. 190° C. (dec.)

### EXAMPLE 1b

2-Amino-5-diethylaminophenylthiosulphonic acid was prepared as above from N,N-diethyl-p-phenylenediamine sulphate, yield=17.32 g (49%), m.p. 196° C. (dec.)

### EXAMPLE 2

### Preparation of Photosensitising Compounds

The arylthiosulphonic acid obtained in example 1 (4 mmol) and alkylaminoalkylaniline (5 mmol) were refluxed in 120 ml methanol and silver carbonate on celite (5 g, 50% w/w) was added slowly over 0.5 h. The reaction mixture was refluxed for a further hour, filtered through a celite pad and the filtrates evaporated. The residue was extracted with dichloromethane and purified by column chromatography on silica.

### EXAMPLE 2a

### 3-(3-Diethylamino-2-hydroxypropyl)(ethyl)amino-7-dimethylaminopheno-thiazinium hydrogensulphate

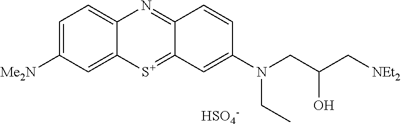

From 2-amino-5-dimethylaminobenzenethiosulphonic acid and 1-diethylamino-3-(ethyl(phenyl)amino)propan-2-ol as blue-black powder, yield=258 mg, 13%; m/z, C₂₃H₃₃N₄OS requires 413.24, found 413.21; λₘₐₓ (MeOH) 655 nm. Page 18, line 27

### EXAMPLE 2b

### 3-(3-Trimethoxsilylpropyl)amino-7-dimethylaminophenothiazinium hydrogensulphate

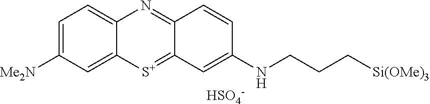

From 2-amino-5-dimethylaminobenzenethiosulphonic acid and N-(3-trimethoxsilyl)propylaniline as a black powder, yield=281 mg, 14%; m/z, C₂₀H₂₈N₃O₃SSi requires 418.60, found 418.62; λₘₐₓ (MeOH) 649 nm.

### EXAMPLE 3

### Preparation of 3,6-Dibromophenothiazinium bromide

10H-Phenothiazine (2 g, 10 mmol) was dissolved in 150 ml of glacial acetic acid at room temperature. Bromine (10 ml, mmol) was added in one amount and the reaction stirred vigorously for one minute. Water (400 ml) was then added in one amount, producing a red-brown solution and a red-black amorphous solid, isolated by filtration at the pump. The solid was washed with water and then ether until the washings were colourless. The resulting dark red solid was powdered and dried in vacuo at room temperature to constant weight, yield=4.96 g, 84%.

### EXAMPLE 4

### General Synthesis of Symmetrical Photosensitising Compounds

The alkylaminoalkylamino compound (15 mmol) was dissolved in dichloromethane (100 ml) at room temperature, and solid 3,6-dibromophenothiazinium tribromide (1.00 g, 1.7 mmol) was added in one amount. The resulting green-blue solution was stirred at room temperature for a further two hours at room temperature. Product isolation was achieved by aqueous washing of the reaction mixture, drying and concentration of the resulting organic solution and precipitation with dry ether. Further purification by column chromatography, (SiO₂/CH₂Cl₂, CH₃OH), was sometimes necessary. Using this method, the following compounds were prepared:

### EXAMPLE 4a

### 3,7-Bis(bis(3-(dimethylamino)propyl)amino)phenothiazinium bromide

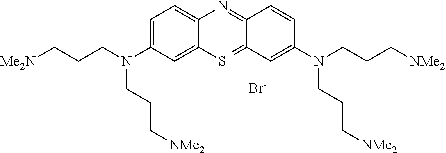

From bis(3-(dimethylaminopropyl)amine, as a blue black solid, yield=228 mg, 21%; m/z, C₃₂H₅₄N₇S requires 568.42, found 568.4; λₘₐₓ (MeOH) 668 nm.

### EXAMPLE 4b

### 3,7-Bis(bis(3-(diethylamino)propyl)amino)phenothiazinium bromide

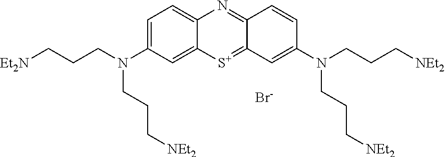

From bis(3-(diethylaminopropyl)amine, as a blue black solid, yield=396 mg, 31%; m/z, C₄₀H₇₀N₇S requires 680.54, found 680.51; λₘₐₓ (MeOH) 667 nm.

### EXAMPLE 4c

### 3,7-Bis((3-(dimethylamino)propyl)(methyl)amino)phenothiazinium bromide

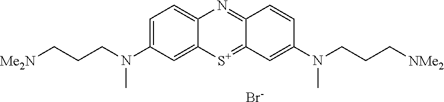

From (3-(dimethylamino)propyl)methylamine as a blue black solid, yield=161 mg, 19%; m/z, C₂₄H₃₆N₅S requires 426.27, found 426.29; λₘₐₓ (MeOH) 662 nm.

### EXAMPLE 4d

### 3,7-Bis((2-(diethylamino)ethyl)(ethyl)amino)phenothiazinium bromide

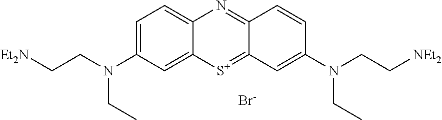

From (2-(diethylamino)ethyl)ethylamine as a blue black solid, yield=189 mg, 20%; m/z, C₂₈H₄₄N₅S requires 482.33, found 482.3; λₘₐₓ (MeOH) 663 nm.

### EXAMPLE 4e

### 3,7-Bis((2-(dimethylamino)ethyl)(ethyl)amino)phenothiazinium bromide

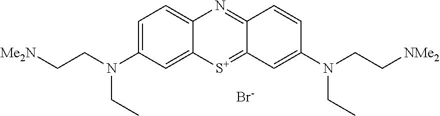

From (2-(dimethylamino)ethyl)ethylamine as a blue black solid, yield=187 mg, 22%; m/z, C₂₈H₄₄N₅S requires 426.27, found 426.28; λₘₐₓ (MeOH) 662 nm.

### EXAMPLE 4f

### 3,7-Bis((2-(diethylamino)ethyl)(methyl)amino)phenothiazinium bromide

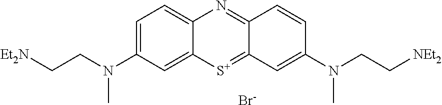

From (2-(dimethylamino)ethyl)methylamine as a blue black solid, yield=206 mg, 23%; m/z, C₂₆H₄₀N₅S requires 454.3, found 454.3; λₘₐₓ (MeOH) 663 nm.

### EXAMPLE 5

### Preparation of Phenothiazinium Tetraiodide

10H-Phenothiazine (2 g, 10 mmol) was dissolved in 50 ml of dichloromethane at room temperature. A solution of iodine (8 g, 32 mmol) in dichloromethane (150 ml) was added, and the whole stirred for three hours at room temperature. The resulting purple-black solid was filtered at the pump, washed free of iodine with dichloromethane, powdered and dried to constant weight. Yield of black powder=6.05 g, 86%.

### EXAMPLE 6

### Preparation of 3-Dialkylaminophenothiazinium triiodides

Phenothiazinium tetraiodide (2.15 g, mmol) was dissolved in methanol (20 ml) at room temperature and a solution of dialkylamine (7.6 mmol) in methanol (20 ml) added dropwise over 20 minutes. The reaction was allowed to stir at room temperature for a further three hours, monitored by thin layer chromatography (SiO₂/3% aqueous NH₄OAc in CH₃OH). The solution was allowed to stand overnight and the resulting black solid filtered off and washed with cold methanol.

### EXAMPLE 7

### Preparation of 3-Dialkylamino-7-dialkylaminoalkylaminophenothiazinium iodides

To a suspension of 3-dialkylaminophenothiazinium triiodide prepared according to example 6 (0.75 mmol) in methanol (10 ml) was added dropwise the requisite dialkylaminoalkylamine (1.8 mmol) in 10 ml methanol. The reaction was allowed to stir at room temperature for 3 hours and then to stand overnight. The resulting black solid was filtered at the pump, dried and then recrystallised from the minimum of methanol. Several examples required further purification by column chromatography, (SiO₂/CH₂Cl₂, CH₃OH). Using this method, the following compounds were prepared:

### EXAMPLE 7a

### 3-(Diethylamino)-7-(5-(diethylamino)pentan-2-ylamino)phenothiazinium iodide

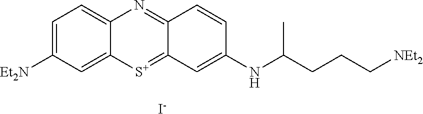

From 3-diethylaminophenothiazinium triiodide and 5-(diethylamino)-2-aminopentane as a black solid, yield=83 mg, 20%; m/z, C₂₅H₃₇N₄S requires 425.27, found 425.27; λₘₐₓ (MeOH) 650 nm.

### EXAMPLE 7b

### 3-(Di-n-hexylamino)-7-((2-(diethylamino)ethyl)(ethyl)amino)phenothiazinium iodide

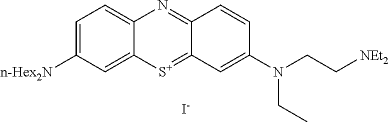

From 3-di-n-hexylaminophenothiazinium triiodide and (2-(diethylamino)ethyl)ethylamine as a black solid, yield=171 mg, 35%; m/z, C₃₂H₅₁N₄S requires 523.38, found 523.38; λₘₐₓ (MeOH) 660 nm.

### EXAMPLE 7c

### 3-(Di-n-butylamino)-7-((2-(diethylamino)ethyl)(ethyl)amino)phenothiazinium iodide

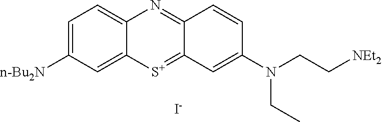

From 3-di-n-butylaminophenothiazinium triiodide and (2-(dimethylamino)ethyl)ethylamine as a black solid, yield=129 mg, 29%; m/z, C₂₈H₄₃N₄S requires 467.32, found 467.28; λₘₐₓ (MeOH) 665 nm.

### EXAMPLE 7d

### 3-(Diethylamino)-7-((2-(diethylamino)ethyl)(methyl)amino)phenothiazinium iodide

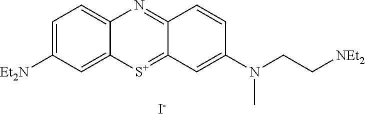

From 3-diethylaminophenothiazinium triiodide and (2-(diethylamino)ethyl)methylamine as a black solid, yield=102 mg, 26%; m/z, C₂₃H₃₃N₄S requires 397.24, found 397.20; λₘₐₓ (MeOH) 652 nm.

### EXAMPLE 7e

### 3-(Bis(3-(dimethylamino)propyl)amino)-7-(di-n-propylamino)phenothiazinium iodide

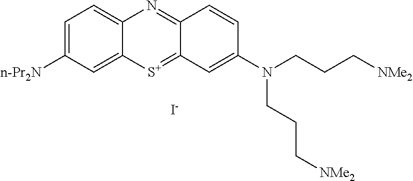

From 3-di-n-propylaminophenothiazinium triiodide and bis(3-(dimethylaminopropyl)amine as a black solid, yield=151 mg, 33%; m/z, C₂₈H₄₄N₅S requires 482.33, found 482.29; λₘₐₓ (MeOH) 661 nm.

### EXAMPLE 7f

### 3-(bis-(2-hydroxyethyl)amino)-7-(3-(1-morpholino)propylaminophenothiazinium iodide

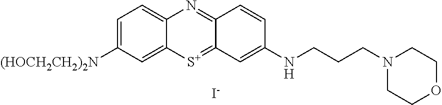

From 3-di-n-propylaminophenothiazinium triiodide and bis(3-(dimethylaminopropyl)amine as a black solid, yield=77 mg, 18%; m/z, C₂₈H₄₄N₅S requires 443.21, found 443.23; λₘₐₓ (MeOH) 661 nm.

### EXAMPLE 7g

### 7,7′-(4,4′-(ethane-1,2-diyl)bis(piperazine-4,1-diyl))bis(3-(dibutylamino)phenothiazin-5-ium)iodide

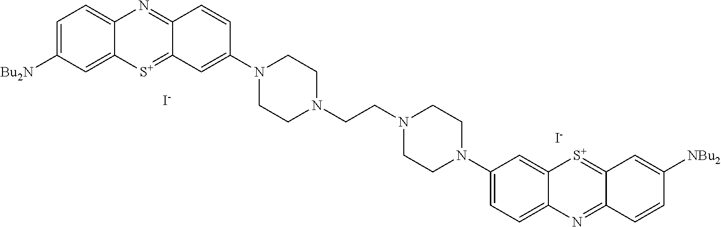

From 3-di-n-butylaminophenothiazinium triiodide (0.14 mmol) and 1,2-bis(piperazin-1-yl)ethane (0.07 mmol) as a black solid, yield=15 mg, 19%; m/z, C₅₀H₆₈N₈S₂ requires 845.26, found 845.40; λₘₐₓ (MeOH) 674 nm.

### EXAMPLE 8

### Singlet Oxygen Production

Singlet oxygen production by the compounds obtained in examples 2, 4 and 7 was assayed using the decolourisation of 2,3,4,5-tetraphenylcylopentadienone (TPCPD) in dichloromethane. Thus the decrease in absorption at 500 nm was monitored spectrophotometrically with time using the method described by Cincotta et al. in “Novel red absorbing benzo[α]phenoxazinium and benzo[α]phenothiazinium photosensitizers: in vitro evaluation”, Photochem. Photobiol. 46 (1987) 751-758. The singlet oxygen yield for the standard photosensitiser, methylene blue (Φ_{ΔMB}) is given as 0.443. By assuming that the decrease in absorption of TPCPD at 500 nm is directly proportional to its reaction with singlet oxygen, the time for a 50% decrease in absorption caused by each of the derivatives under identical conditions (t_{1/2Der}) thus gives a measure of its photosensitising efficiency. Thus, the time for the TPCPD absorption to decrease by 50% due to MB photosensitisation (t_{1/2MB}) was taken as 1.0. To calculate the singlet oxygen yield for the derivative compounds of the present invention (Φ_{ΔDer}), the following formula was used:

$${Φ}_{Δ Der} = {Φ}_{Δ MB} \cdot \frac{{t}_{1 / 2 MB}}{{t}_{1 / 2 Der}}$$

The singlet oxidation yield for the compounds prepared in examples 7a, 7d, 4b, 4f, 7c, 7b and 7f are given in table 1 below.

TABLE 1

| Compound from | | | |
|---|---|---|---|
| example number | λₘₐₓ (MeOH, nm) | LogP | Relative Φ_{Δ} |
| MB | 656 | −0.10 | 1.00 |
| 7a | 650 | +0.65 | 0.2 |
| 7d | 652 | +0.74 | 1.4 |
| 4b | 667 | +0.11 | — |
| 4f | 663 | +0.27 | 0.6 |
| 7c | 665 | +1.09 | 0.3 |
| 7b | 660 | +1.25 | 0.4 |
| 7f | 661 | +0.57 | 0.2 |

### EXAMPLE 9

### Hydrophilic-Lipophilic Balance (Log P)

The lipophilicities of the photosensitisers were calculated in terms of log P, the logarithm of their partition coefficients between phosphate-buffered saline and 1-octanol. The data were calculated using the standard spectrophotometric method [ref] based on the relationship:

$$Log P = Log \{ \frac{( A - {A}^{1} )}{{A}^{1}} \cdot \frac{{V}_{W}}{{V}_{O}} \}$$

where A and A¹ are the absorption intensities before and after partitioning respectively and V_{w} and Vₒ are the respective volumes of the aqueous and 1-octanol phases. Determinations were repeated three times.

The Log P values for the compounds prepared in examples 7a, 7d, 4b, 4f, 7c, 7b and 7f are given in table 1 above.

### EXAMPLE 10

### Antimicrobial Screening

The photobactericidal efficacies of the derivatives in addition to that of the known photosensitiser methylene blue were measured against both Gram positive Staphylococcus aureus (NCTC 6571) and Enterococcus faecalis (NCIMB 13280) and Gram negative Escherichia coli (NCTC 10418), Proteus mirabilis (NCIMB 5887) and a clinical strain of Klebsiella pneumoniae bacteria. Both strains were grown in Mueller-Hinton Broth and then diluted to a concentration of 10⁶ colony-forming units/ml. Aliquots of the strains were then incubated for 1 hour at 37° C. in microtitre trays with various concentrations of photosensitiser in a range of from 100 to 3 μM, with zero photosensitiser concentrations in each case for control purposes. The trays were then either illuminated for twenty minutes using an array of light-emitting diodes (660 nm) giving a light dose of 6.2 J cm⁻² or alternatively foil-covered to provide dark controls. From each well showing an inhibition of growth of the micro-organism, 1 μl was sub-cultured on nutrient agar, using the Miles-Misra method, and incubated for 18 hours at 37° C. The minimum bactericidal concentrations were then determined as the lowest concentration for each photosensitiser giving no bacterial growth.

Antifungal screening was carried out similarly against the yeast Candida albicans (NCPF 8179), using Sabouraud broth and agar.

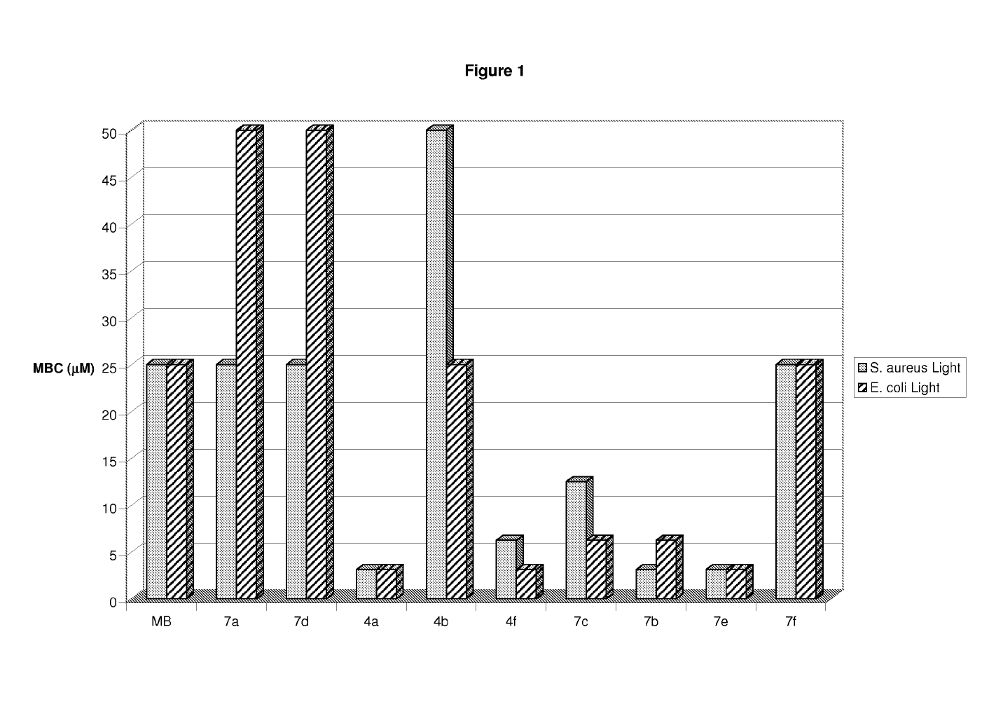

Antimicrobial results are shown in Tables 2 and 3, and in FIG. 1.

TABLE 2

Minimum Bactericidal Concentration (μM)

| Example | S. aureus | S. aureus | E. coli | E. coli |
|---|---|---|---|---|
| number | (Light) | (Dark) | (Light) | (Dark) |
| Methylene blue | 25 | | 25 | |
| 7a | 25 | 100 | 50 | 100 |
| 7d | 25 | 50 | 50 | 100 |
| 4a | 3.125 | 6.25 | 3.125 | 12.5 |
| 4b | 50 | 100 | 25 | 100 |
| 4f | 6.25 | >100 | 3.125 | >100 |
| 7c | 12.5 | 25 | 6.25 | 25 |
| 7b | 3.125 | 12.5 | 6.25 | 25 |
| 7e | 3.125 | >100 | 3.125 | >100 |
| 7f | 25 | 25 | 25 | 50 |

TABLE 3

| | Minimum bactericidal/fungicidal concentration (μM) | | | | | | | |
|---|---|---|---|---|---|---|---|---|
| | | | | | K. | | | |
| Example | E. faecalis | | P. mirabilis | | pneumoniae | | C. albicans | |
| number | Light | Dark | Light | Dark | Light | Dark | Light | Dark |
| 7b | 0.19 | 100 | 0.39 | 100 | 0.19 | 100 | 3.13 | 25 |
| 2b | 0.78 | 50 | 1.56 | 100 | 1.56 | 100 | 3.13 | 25 |
| 7g | 1.56 | 12.5 | 0.78 | 100 | 3.13 | 100 | 3.13 | 25 |
| 7c | 0.39 | 6.25 | 0.78 | 100 | 6.25 | 100 | 3.13 | 25 |
| 7e | 0.19 | 100 | 0.39 | 100 | 3.13 | 100 | 3.13 | 100 |

1. A compound of formula (II): 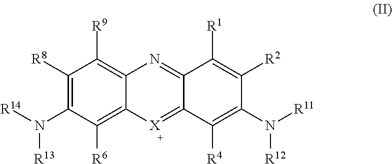
or a pharmaceutically acceptable derivative thereof for use in a method of combating and/or detecting a pathogen;
wherein X is selected from O, S and Se; each of R¹, R², R⁴, R⁶, R⁸, R⁹, R¹¹, R¹², R¹³ and R¹⁴ is independently selected from hydrogen, halogen, sulfo, sulfoxy, mercapto, acyl, nitro, amino or an optionally substituted alkyl, alkenyl, alkynyl, aryl, amine or alkoxy group; provided that at least one of R¹¹, R¹², R¹³ and R¹⁴ is an optionally substituted amine group.

2. A compound for use in a method of combating and/or detecting a pathogen according to claim 1 wherein X is sulfur.

3. A compound for use in a method of combating and/or detecting a pathogen according to claim 1 wherein each of R¹, R², R⁸ and R⁹ is independently selected from hydrogen, an optionally substituted alkyl group or an optionally substituted alkoxy group.

4. A compound for use in a method of combating and/or detecting a pathogen according to claim 1 wherein each of R⁴ and R⁶ is independently selected from hydrogen and halogen.

5. A compound for use in a method of combating and/or detecting a pathogen according to claim 1 wherein the pathogen is selected from viruses, bacteria, fungi and protozoal infections.

6. A compound for use in a method of combating and/or detecting a pathogen according to claim 1 wherein the method comprises exposing the compounds to light having a wavelength of 500 to 900 nm in the presence of oxygen.

7. A compound for use in a method of detecting a pathogen according to claim 1 wherein X is oxygen.

8. A composition comprising a compound of formula (II) as defined in claim 1 and a pharmaceutically acceptable carrier.

9. A compound of formula (II): 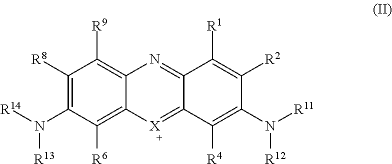
or a pharmaceutically acceptable derivative thereof;
wherein X is selected from O, S and Se; each of R¹, R², R⁴, R⁶, R⁸, R⁹, R¹¹, R¹², R¹³ and R¹⁴ is independently selected from hydrogen, halogen, sulfo, sulfoxy, mercapto, acyl, nitro, amino or an optionally substituted alkyl, alkenyl, alkynyl, aryl, amine or alkoxy group; provided that at least one of R¹¹, R¹², R¹³ and R¹⁴ is an optionally substituted amine group;
other than compounds having the formula Va to Ve:

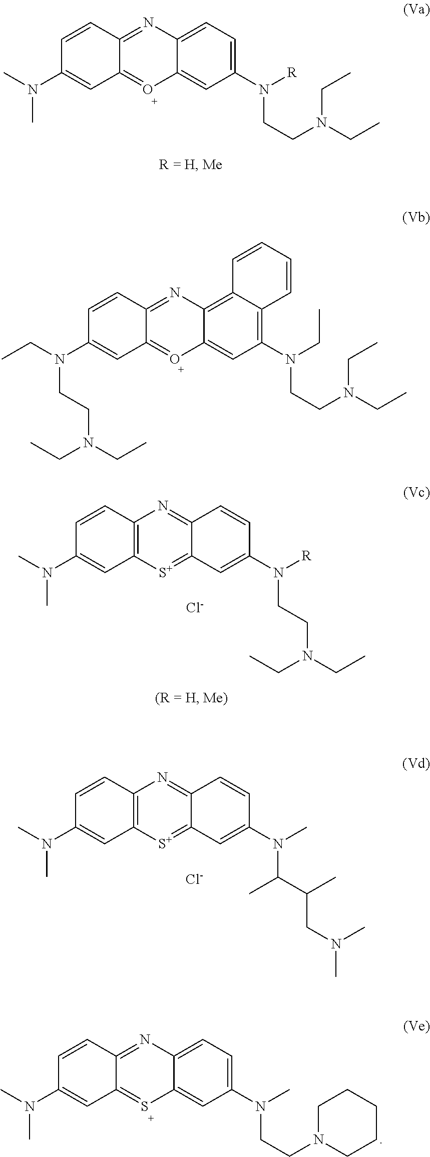

10. A compound as claimed in claim 9 having one of the following structures:

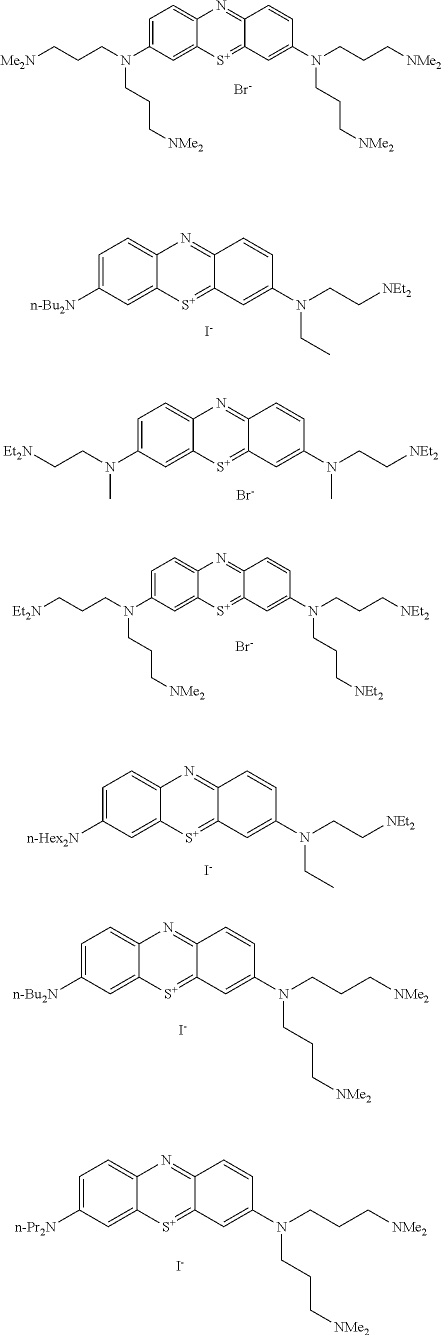

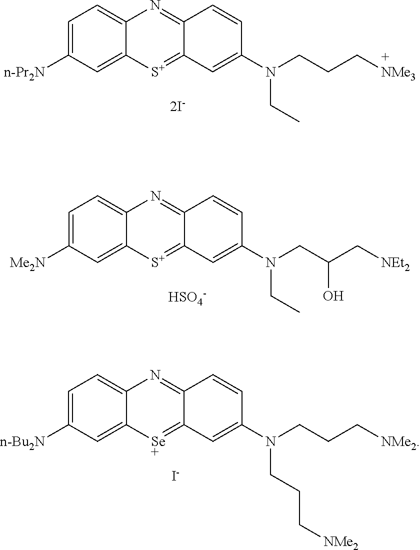

11. A method of preparing a compound of formula (II) as defined in claim 1, the method comprising reacting a phenothiazinium compound of formula (VII): 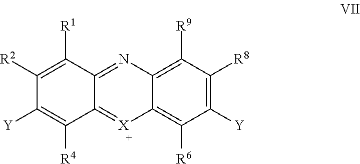
with an amine of formula R¹³R¹⁴NH and an amine of formula R¹¹R¹²NH;
wherein R¹, R², R⁴, R⁶, R⁸, R⁹, R¹¹, R¹², R¹³ and R¹⁴ are as defined in claim 1 and each Y is independently a halogen or hydrogen.

12. A method of treating an animal having a disease caused by a pathogen, the method comprising administering to the animal a compound of formula (II) as defined in claim 1.

13. A method of combating and/or detecting a pathogen at a locus, the method comprising applying to the locus a composition comprising a compound of formula (II) as defined in claim 1; and exposing the locus to light having a wavelength of from 500 to 900 nm.
